(19) United States
(12) Patent Application Publication
(10) Pub. No.: US 2002/0028847 A1
(43) Pub. Date: Mar. 7, 2002

(54) Method for treatment of fibrosis related diseases by the administration of prostacyclin derivatives

(75) Inventors: Richard Stratton, London (GB); Carol Black, London (GB); George R. Martin, Palo Alto, CA (US); David F. Carmichael, Pacifica, CA (US)

Correspondence Address: ROBINS & PASTERNAK LLP, Suite 200, 90 Middlefield Road, Menlo Park, CA, 94025, US

(73) Assignee: Fibrogen, Inc.

(21) Appl. No.: 09/915,947

(22) Filed: Jul. 25, 2001

Related U.S. Application Data
(63) Continuation of application No. 09/349,516, filed on Jul. 8, 1999, now abandoned.
(60) Provisional application No. 60/092,044, filed on Jul. 8, 1998.

(51) Int. Cl. A61K 31/5585

(52) U.S. Cl. 514/559; 514/461

(57) ABSTRACT

The present invention is directed to methods for treating fibrosis related diseases and disorders, particularly scleroderma by treating a patient in need with a pharmaceutically efficacious amount of a prostacyclin derivative. The most preferred prostacyclin derivatives are cicaprost and iloprost.

### CROSS REFERENCE TO RELATED APPLICATIONS

This application is a continuation-in-part of U.S. Provisional Application Ser. No.: 60/092,044, filed, Jul. 8, 1998.

### BACKGROUND OF THE INVENTION

Fibrosis Related Diseases And Disorders. The deposition of excess connective tissue is found in a variety of diseases and disorders. These diseases and disorders has been designated as “fibrosis-related” disorders and are implicated in over 56% of the deaths in the United States and a comparable percentage worldwide. Fibrosis-related disorders include, excessive scarring, fibrosis of the internal organs (e.g. liver cirrhosis), and scleroderma.

Scleroderma is a connective tissue disease characterized by the deposition of excess collagen in skin and internal organs. Various estimates of the incidence of this disease worldwide suggest that approximately four- to twelve-million patients are afflicted with some form of scleroderma (Medsger & Masi, 1971, Ann. Intern. Med. 74:714-721), although hospital based studies may underestimate the true incidence of the condition by failing to record mild cases of the disorder that are treated for by general practioners.

Almost 100% of the individuals diagnosed with scleroderma also suffer from Raynaud's phenomena, a transient disturbance of the peripheral circulation. In contrast, however, critical digital ischaemia, while prevalent in scleroderma patients, is only rarely seen in patients with the primary form of Raynaud's phenomena. Stratton & Black, 1997, Critical Ischaemia, 6:69-73.

Connective Tissue Growth Factor. The expression of Connective Tissue Growth Factor (CTGF), as evidenced by the existence of CTGF mRNA, has been implicated in the development of fibrosis related diseases and disorders. See e.g., Igarishi, et al., 1996, J. Invest. Dermatology 106:729-733. For example, as reported in Igarishi, et al., CTGF MRNA is strongly expressed by cutaneous fibroblasts in lesional skin biopsy material from sclerodema patients, but not in surrounding healthy tissue. Current concepts suggest that CTGF is an autocrine cytokine that stimulates and sustains scarring and fibrotic reactions.

Prostacyclin Derivatives. Prostacyclin derivatives that are chemically stable and highly pharmacologically potent have been reported. See, e.g. U.S. Pat. No. 4,692,464; Hildebrand, 1994, Prostaglandins 48(5): 297-312; and Hildebrand M, 1992, Prostglandins 44(5):431. Among these prostacyclin derivatives are the compounds, cicaprost and iloprost, whose pharmacological and pharmacokinetic profiles have been characterized in a number of animal species and in humans as described in, for example, Hildebrand M., 1992, inter alia.

Prostacyclin derivatives have been shown to inhibit specifically the formation of metastasis in experimental tumor models. In particular, cicaprost has been previously shown to effectively inhibit metastasis in several different animal models. See e.g., Schneider et al., 1994, Cancer Metastasis Review 13:349-364. Cicaprost is metabolically stabilized by the introduction of an oxygen atom at position 3 of the pentanoic acid chain, preventing beta-oxidation. Both cicaprost and the pro-drug, eptaloprost, have been demonstrated to be antimetastically acting agents. See, e.g., U.S. Pat. No. 5,545,671.

Iloprost is a synthetic prostacyclin analogue and is described in, for example, U.S. Pat. No. 5,663,203 (issued Sep. 2, 1997). Specifically, Iloprost bears the systematic designation 5-(E)-(1S,5S,6R)-7-hydroxy-6[(E)-(3S,4RS)-3-hydroxy-4-methyl-1-octen-6-inyl]bicyclo[3.3.0]octen-3-ylidene pentanoic acid.

Iloprost has been shown to prevent platelet thrombus formation in animals with thrombin induced thrombosis. Shonberge, et al, 1995, J. Lab. Clin. Med. 125:96-101. Iloprost, as well as other members of the PGI2 family of compounds, reportedly possess high inhibition activity of platelet aggregation and high stimulatory activity of vasodilating angiotelectasis such that the compounds may be helpful as a treatment against peripheral blood circulation impairments. See, U.S. Pat. No. 5,679,707 (issued Oct. 21, 1997) (method for treating hemorrhoids), U.S. Pat. No. 5,703,099 (issued Dec. 30, 1997) (describing a novel compound having an activity of PGI2 receptor agonist and the activity of such compound), U.S. Pat. No. 5,654,339 (issued Aug. 5, 1997) (describing the use of Iloprost as a pharmaceutical agent for the treatment of chronic polyarthritis).

Iloprost has been shown to be safe and effective therapy for patients with Raynaud's phenomena (Kyle, et al, 1992, J. Rheumatol. 19:1403-06; see also, U.S. Pat. No. 5,663,203 (issued Sep. 2, 1997) (describing topical application of PGI2 inhibitors for the treatment of Reynaud's phenomena) and reportedly may be of benefit in scleroderma-associated pulmonary hypertension (de la Mata, et al., 1994, Arthritis Rheum 37:1528-33). As reported in Mascagni, et al., 1996, Fourth International Workshop On Scleroderma Research (Abstract), p. 25, a reduction of the resistive index of the renal interlobar and cortical arteries for scelroderma patients receiving iloprost infusions has also been observed.

Observations related to the reduction of cytokine production by the peripheral blood mononuclear cells and improved skin fibrosis conditions following administration of iloprost to patients with scleroderma suggest that Iloprost may be of use in treating scleroderma. Notwithstanding the related art, prior to the present invention, however, no direct correlation between Iloprost and its anti-fibrotic properties has been measured.

### SUMMARY OF THE INVENTION

The present invention is directed to methods of treating fibrosis-related diseases and disorders. More particularly, the present invention is directed to a method for treating and ameliorating the fibrosis occurring in scleroderma. The present invention describes methods and treatments that are directed to modulating the excessive production of connective tissue in patients having scleroderma and modulating the production of such excess connective tissue in scleroderma patients by the administration of a therapeutically effective amount of a prostacyclin derivative. More particularly, the present invention is directed to methods and treatments for ameliorating the fibrosis-related symptoms of scleroderma by modulating the production of excess connective tissue by inhibiting the activity of CTGF and its related factors, including TGF-β.

In a preferred embodiment of the present invention, the methods of the present invention are directed to the administration of cicaprost or iloprost delivered either systemically through intravenous administration or orally.

### DETAILED DESCRIPTION OF THE INVENTION

### A. BRIEF DESCRIPTION OF THE FIGURES

FIGS. 1 and 2 (FIG. 1 and FIG. 2) set forth results from a Northern Blot analysis, measuring the presence of CTGF RNA in untreated and treated cells. TGF-β, a profibrotic cytokine, induces the synthesis of CTGF in RNA and this response is blocked in cells treated with iloprost.

### B. DESCRIPTION OF THE INVENTION

1. Methods Of Treatment

The present invention is directed to a method for treating and ameliorating fibrosis, in particular, those related to symptoms of scleroderma. More particularly, the present invention describes methods and treatments that are directed to modulating the excessive production of connective tissue in patients having scleroderma and other fibrosis related diseases by the administration of a prostacyclin derivative. Prostacyclin derivatives used according to the methods of the present invention include carbacyclin derivatives as described in U.S. Pat. No. 4,692,464. Other suitable prostacyclin derivatives that may be used in the methods of the present invention are disclosed in U.S. Pat. Nos. 4,191,694; 4,219,479; 4,315,013; 4,364950; 4,378,370; 4,466,969, in each case, suitable species being routinely selected in accordance with their ability to modulate the production of excess connective tissue by inhibiting the activity of CTGF and its related factors, including TGF-β. In a most preferred embodiment, the prostacyclin derivatives used in the methods of the present invention are cicaprost and iloprost.

The present invention relates to the treatment of fibrosis-related diseases, including liver cirrhosis, kidney fibrosis, and more particularly scleroderma. The methods of the present invention involves administering to a subject in need a therapeutically effective amount of a prostacyclin derivative, more particularly the compounds, cicaprost and iloprost. Cicaprost may be produced according to the processes described in, for example, U.S. Pat. No. 4,692,464 and 4,886,788. Methods of producing iloprost are described in, for example, EP 11591 and U.S. Pat. No. 4,692,464.

Although the utility of iloprost to treat the symptoms of Reynaud's phenomena in scleroderma patients was previously disclosed, the ability of iloprost to treat the symptoms of scleroderma related to the excess deposition of connective tissue was not previously disclosed. As described herein, iloprost is capable of inhibiting collagen production and CTGF production in sclerotic cells. As set forth in the art, collagen production and the precursor production of CTGF, a growth factor implicated in the production of collagen, are inexorably tied to the production of fibrotic conditions. The utility of iloprost to inhibit collagen and CTGF production was not known until the present invention. More specifically, the utility of iloprost to treat the fibrotic conditions associated with scleroderma was not known. As set forth for the first time in this Specification, iloprost is capable of interfering with the mechanism by which collagen is formed in scleroderma patients, in part, by inhibiting the activity of CTGF.

Even more particularly, the present invention is directed to methods and treatments for ameliorating the fibrosis-related symptoms of scleroderma by modulating the production of excess connective tissue by inhibiting the activity of CTGF and related factors, including TGF-β.

2. Pharmaceutical Formulations and Routes of Administration

The prostacyclin derivative such as cicaprost or iloprost may be administered to a patient, by itself, or in pharmaceutical compositions where it is mixed with suitable carriers or excipient(s) at doses to treat or ameliorate a variety of fibrosis related diseases and disorders, and particularly scleroderma. Techniques for formulation and administration of the compounds of the instant application may be found in “Remington's Pharmaceutical Sciences,” Mack Publishing Co., Easton, Pa., latest edition.

a. Routes of Administration

Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections.

Alternately, one may administer the compound in a local rather than systemic manner, for example, in a depot or sustained release formulation.

b. Composition/Formulation

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated.

Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration,the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A preferred pharmaceutical carrier for the hydrophobic compounds of the invention is a cosolvent system comprising benzyl alcohol, a nonpolar surfactant, a water-miscible organic polymer, and an aqueous phase. A preferred cosolvent system is the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as DMSO also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeksup to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

C. Effective Dosage

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For any compound used in the method of the invention, the therapeutically effective dose can be estimated initially from cell culture assays. For example, a dose can be formulated in animal models to achieve a circulating concentration range that includes the IC50 as determined in cell culture (i.e., the concentration of the test compound which achieves a half-maximal inhibition of the PTP activity). Such information can be used to more accurately determine useful doses in humans.

A therapeutically effective dose refers to that amount of the compound that results in amelioration of symptoms or a prolongation of survival in a patient. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50ED50. Compounds which exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition. (See e.g. Fingl et al., 1975, in “The Pharmacological Basis of Therapeutics”, Ch. 1 p1).

Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day. Stated in terms of patient body surface areas, usual dosages range from 0.5-1200 mg/m²/day, commonly from 0.5-150 mg/m²/day, typically from 5-100 mg/m²/day.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

d. Packaging

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition. Suitable conditions indicated on the label may include treatment of fibrosis-related diseases, and more particularly, sclerodenna.

### C. EXAMPLES

1. Methods and Protocols for Assays to Determine Collagen Synthesis And CTGF Expression

a. Culture of Human Skin Fibroblasts

4 mm punch skin biopsies were taken from the forearms of healthy volunteers and from patients with active diffuse scleroderma. Fibroblasts from these biopsies were subcultured to passage 6 in Dulbecco's modified eagle medium with 10% fetal calf serum and Pen Strep solution and grown to confluence. Twenty-four hours before assay, the fibroblasts were switched to serum free media with exchange of media every eight (8) hours. Upon switching to serum free medium (L-ascorbic acid phosphate magnesium salt n-hydrate) (C₆H₆O₉P) 3/2 Mg was added at a final concentration of 30 μg/ml.

b. ELISA for Collagen (N propeptide of Type I Collagen α Chain)

The supernatant fluid from the above described culture was diluted 1/100 in 0.1 M NaHCO₃ and 100 μl of said diluted culture was added to each well of a 96 well maxisorp plate. The plate was then left at room temperature for two hours on a plate shaker. The plate was then washed once in PBS. 200 μl of 2% BSA in PBS was then added and the plate was left for two hours to block non-specific binding.

The plate was then washed four times in PBS and first layer antibody, anti-N-procollagen I rabbit polyclonal, diluted 1/2000 in 1% BSA PBS, was added at 100 μl/well. The plate was left for two (2) hours at room temperature and then washed four times in PBS. A second layer antibody, HRP labeled goat anti-rabbit IgG 1/2000, was then added and left for two hours. The plates was then washed again four times in PBS. OPD (one tablet—in freezer—of OPD, 25 ml OPD buffer and 10 μl of 30% H₂O₂) was then added and the process was stopped with 50 μl 3M NHCl. The plate was then read on a multiwell analyser.

C. Western Blot for CTGF

50 μl of heparin coated Sepharose beads were used in the Western Blot described herein. 1.5 ml of supernatant was added to the beads and the beads were then resuspended and left on a shaker overnight. The suspension was washed three times in PBS and resuspended in sample buffer 100 μl at 100° C. for 10 minutes and loaded on 10-20% tris/glycine gel (Novex). More specifically, a Tris/glycine running buffer, non reducing sample buffer was used and the gel was loaded at 20 μl per well and run at 135 volts 12 mA per plate for 2-3 hours. The gel was then removed from its plastic case and laced in a 20% methanol transfer buffer as follows: (1) +ive; (2) felt pad (2×); (3) nitrocellulose; (4) gel; (5) gel; (6) felt pad (2×); (7) −ive. The transferred protein was then placed in a Minigel chamber at 30 volts for 1½ hours and then removed and blocked in PBSa 5% marvel milk for two hours. First layer anti TGF-β at 1/1000 was added and then washed four times in blocking solution. A second layer HRP conjugated antibody 1/1000 was added and then washed four times in blocking solution and then washed two times in PBSa. Staining was accomplished using chemiluminesence solution (5 ml per nitrocellulose sheet, Pierce super signal chemiluminesence substrate number 34080). The sheets were placed in darkroom cannisters under cling film with orientation markers and then developed on photographic plates.

d. CTGF ELISA Protocol

The following protocol was used for the CTGF ELISA:

1. Plate antigen on Nunc immuno plase Maxisorp surface (catalog number 442402).

2. CTGF standard curve by two fold serial dilution in 6 triplicates (see Table 1, below).

3. Add 50 μl of sample to each well.

4. Leave wells for 2 hours at room temperature.

5. Wash wells 4 times with PBSa.

6. Add first layer antibody (pAB2 rabbit anti-human CTGF (0.8 mg/ml)(1/1280)) diluted to 625 ng/ml in blocking buffer (1% BSA/0.05% tween20/PBS).

7. Add 50 μl to each well and leave at room temperature for one hour.

8. Wash wells five times with PBS.

9. Add second layer antibody donkey anti-rabbit IgG, HRP linked whole antibody (Amersham Life Sciences, Catalog No. NA 934 diluted 1/3200 in block buffer).

10. Add 50 μl per well and leave at room temperature for 30 minutes.

11. Wash wells five times in PBS.

12. Add substrate Gibco BRL TMB ELISA catalog no. 15980-014 at 100 μl per well and incubate for 5 minutes.

13. Stop reaction with 50 μl per well of 1M H₂SO₄ and read OD at 450 nm.

e. Assay for CTGF Promoter Activity by NIH 3T3 Cell Line

The NIH 3T3 cell line was permanently transfected with a CTGF promoter/luciferase construct with an antibiotic resistant gene. The cell line was then plated with TGF-β with variable concentrations of Iloprost. The plates were then left for 24 hours after TGF-β stimulation. The cells were lysed in Promega reporter lysis buffer (E 397A) at 50 μl/well on a 96 well plate at room temperature for five minutes. 100 μl per well of luciferase substrate (Promega E148A) was then added and chemiluminesence was read on a plate reader.

f. Cell Prolieration/Viability Assay

A cell proliferation/viability assay was conducted. This assay measured the conversion of tetrazolium salt (MST-1-Boehringer Mannheim No. 1644807) by mitochondrial dehydrogenases in viable cells, as could be observed by the change of the dye to dark red upon conversion. More specifically, 10 μl of MST-1 per well was added to cells cultured in 100 μl wells. The cells were then cultured for four hours and results were obtained by reading the wells at 450 nm with a reference wavelength of 600 nm.

g. Transfection Protocol

Normal human fibroblasts were grown to 80% confluence in a 6 well plate. 1 μg of DNA (CTGF promoter/luciferase) was added with 3 μl of FuGene 6 transfection reagent (Boehringer Mannheim 1814443); wherein the FuGene 6 was added dropwise to serum free culture medium and left at room temperature for five minutes and added to 1 82 g of DNA in a separate tube for fifteen minutes. The DNA/FuGene 6 solution was then added dropwise to the cell culture by spreading around each well and swirling the flasks. The cell culture was returned to an incubator and the cells were grown again to confluence, approximately 36 hours. The cells were then switched to a serum free medium with ascorbate and left overnight. The cells were then treated for 24 hours as follows: control; Iloprost 1000 pg/ml; TGF-β 20 ng/ml; and TGF-β 20 ng/ml+Iloprost 1000 pg/ml.

After 24 hours the cells were lysed with Promega reporter lysis buffer (E 397A) at room temperature, 400 μl per 100 nm plate and left for 15 minutes at room temperature. A 50 μl sample of the lysate was drawn and 100 μl of luciferase assay reagent (Promega E 148A) was added. Chemiluminensence was read using a plate reader program ARQ.1PT software micro P1.00.

h. Northern Blot Protocol

Cultured cells are grown to confluence on 100 mm plates. The cells were then serum starved with ascorbate for 24 hours, after which conditioned medium with TGF-β and Iloprost was added. The cells were then lysed with TRIzol (Gibco catalog no. 15596-018) wherein 1 ml per plate of the TRIzol was added and the plate was left at room temperature for ten minutes. 200 μl of chloroform was then added and the cells were vortexed for 20 seconds. The cells were then left alone for 5 minutes at room temperature and then centrifuged in a microcentrifuge tube for 10 minutes at maximum speed at 4° C. The upper layer was then removed (layer containing RNA) and then mixed with an equal volume of isopropanol. The mixture was then centrifuged for 10 minutes to form a pellet of RNA. 0.5 ml of 70% ethanol in RNAse free purified water was then added to the pellet. The ethanol was then tipped off and the pellet was left to dry. After five minutes, the pellet was resuspended in 20 μl of RNAse free water and kept on ice. To assay the RNA using a photospectometer, a sample of the RNA was diluted 1/500 (volume 1 ml) and read at OD 260 nm (to measure RNA) and 280 nm (to measure protein). RNA concentration was calculated in terms of μg/μl=(OD260×40×dilution)/1000.

The gel used in the Northern Blot assay was a 1% agarose gel comprised of 1 g agarose in 85 ml RNAse free water which had been microwaved at full power for 2 minutes, allowed to cool down for 1 minute. Following cool down, 10 ml 10×MOPS and 5 ml formaldehyde was added. Following mixing, the gel was poured and allowed to set for 30 minutes. 400 μl deionized formamide, 80 μl 10×MOPS and 140 μl formaldehyde was used as a sample buffer. Specifically, 20 μg RNA was added to 25 μl sample buffer, +3 loading dye and mixed. The mixture was then heated at 100° C. for 2 minutes and then placed on ice. After a few minutes, the mixture was loaded onto the gel. The running buffer used was MOPS-EDTA-Sodium Acetate (Sigma M5755) and the gel was run at 100V for 2-3 hours.

The gel was then transferred to nitrocellulose by placing gel between filter paper 2× and a stack of filter paper wherein the filter paper 2× was saturated with 10×SSC salt solution. The transfer was allowed to take place overnight and transfer was checked by using a UV light. The paper was then baked for 1 hour at 60-80° C. and then washed in a church buffer (1% BSA, 7% SDS, 0.5 M phosphate (pH 7.2), 1 mM EDTA warmed to 65° C.) for 15 minutes.

A probe was prepared as follows: (1) Add 5 μl CTGF cDNA to 40 μl water and heat to 100° C. for 5 minutes; (2) place the solution on ice and transfer to a Rediprime tube containing Rediprime; (3) Add 5 μl of P³²-ctp and incubate the solution at 37° C. for 30 minutes; (3) purify probe using a G-50 column at 1100 rpm for 2 minutes; (4) count CPMS and adjust to a 1 million cpm concentration; (5) heat the probe solution at 100° C. for 5 minutes to denature the cDNA; (6) add blot/church buffer (as described above); (7) roll at 65° C. overnight; (8) wash twice and then count label and background; (9) expose film to blot for two hours at −80° C.; and (10) warm to room temperature and develop film.

2. Results from Assays

The following results were obtained by conducting the above described assays:

1. As set forth below in Tables 2, 3 and 4, comparison of collagen ELISA results regarding collagen production (protocol described at Section C.1.b. above) demonstrate that the excessive production of collagen in scleoderma fibrolasts is inhibited upon administration of Iloprost. As set forth in Table 4 below, five samples were tested and the acronyms set forth along the x-axis of the chart are defined as follows: “C”=control; “I”=Iloprost; “T”=TGF-β; and T+I=TGF-β and Iloprost.

2. As set forth below in Tables 5, 6 and 7, comparison of the CTGF ELISA results (protocol described at Section C.1.d. above) provides evidence that addition of Iloprost to scleroderma fibroblasts results in a decrease in CTGF production, and hence the deposition of collagen. The acronyms used in Table 7 are the same as used for Table 4.

3. The results related to CTGF promoter activity (as described in Section C.1.e, above) are set forth at Table 8. The results related to cell viability (as described in Section C.1.f., above) are set forth at Table 9.

4. As set forth in Table 10 (see, protocol, as set forth above at Section C.1.g.), Iloprost's ability to inhibit CTGF production was measured over a twenty-four hour period. The inhibitory effect of Iloprost was most pronounced between hour 1 and hour 3.

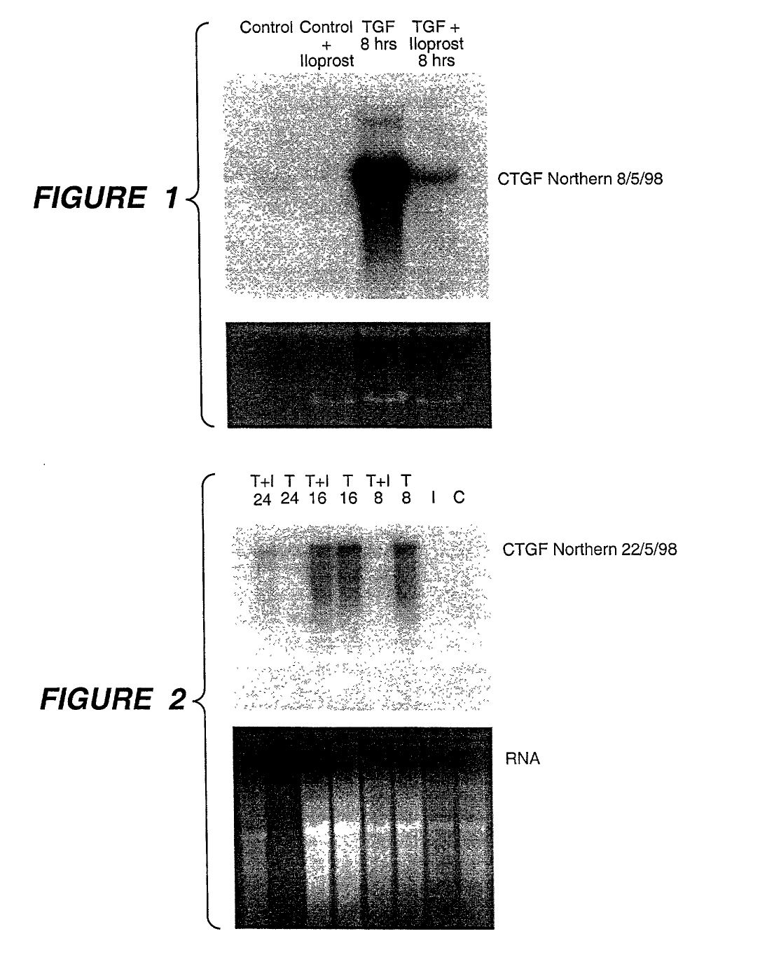

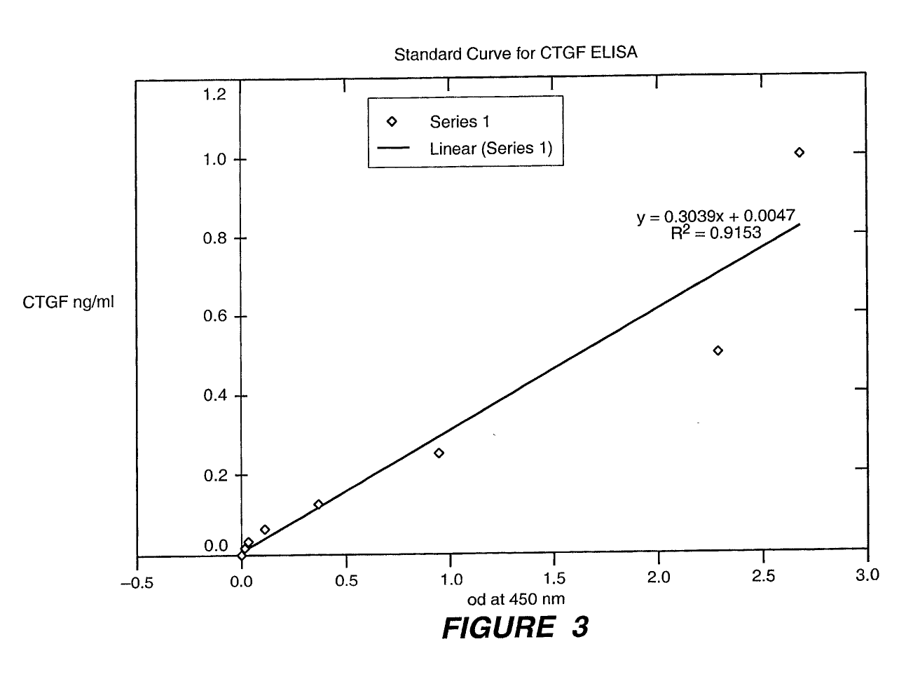

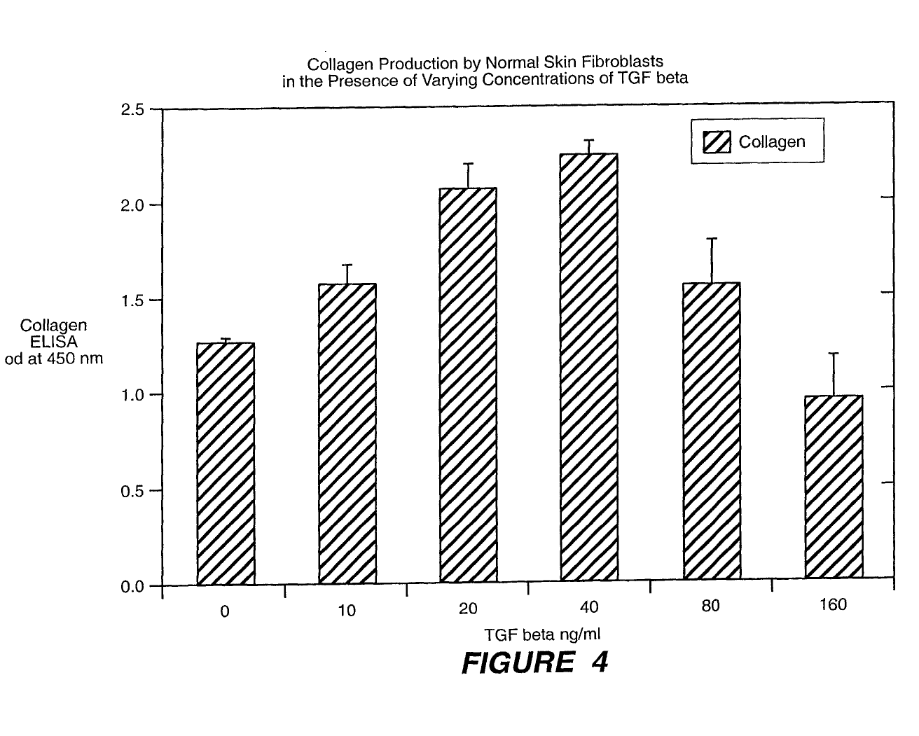

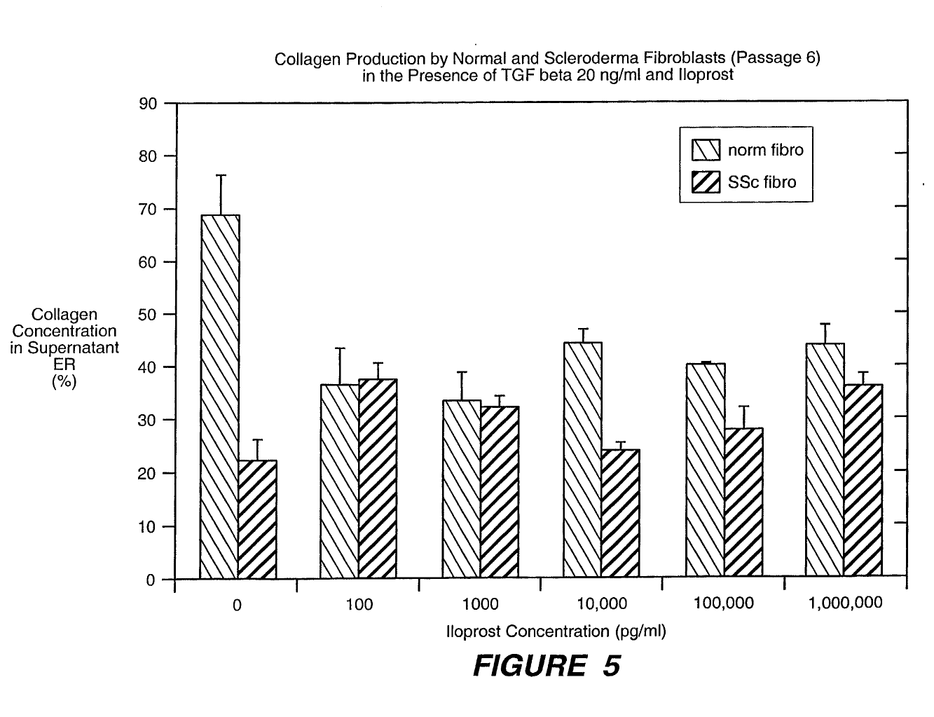

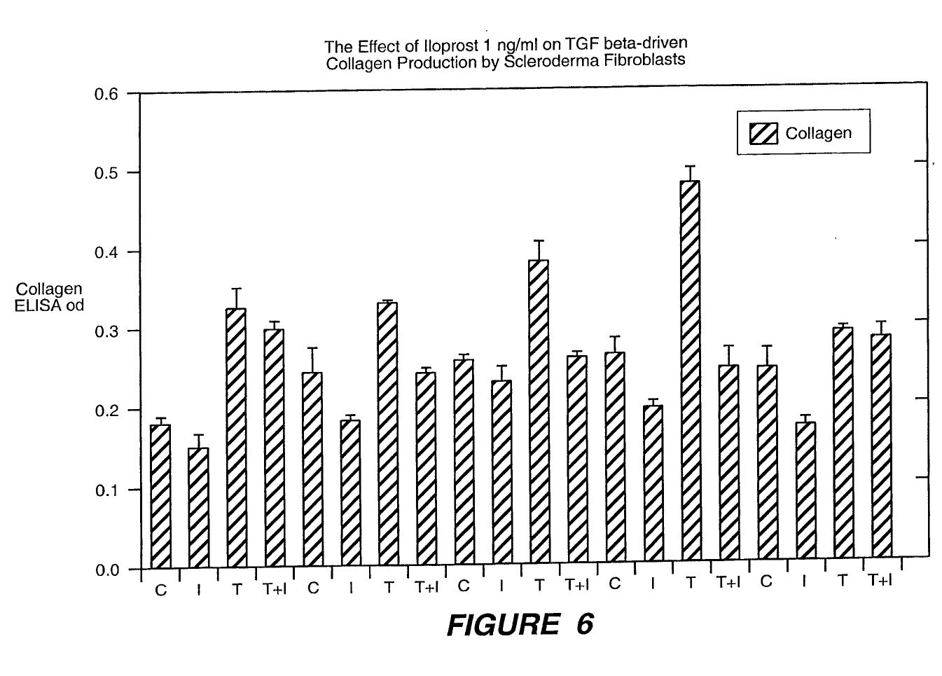

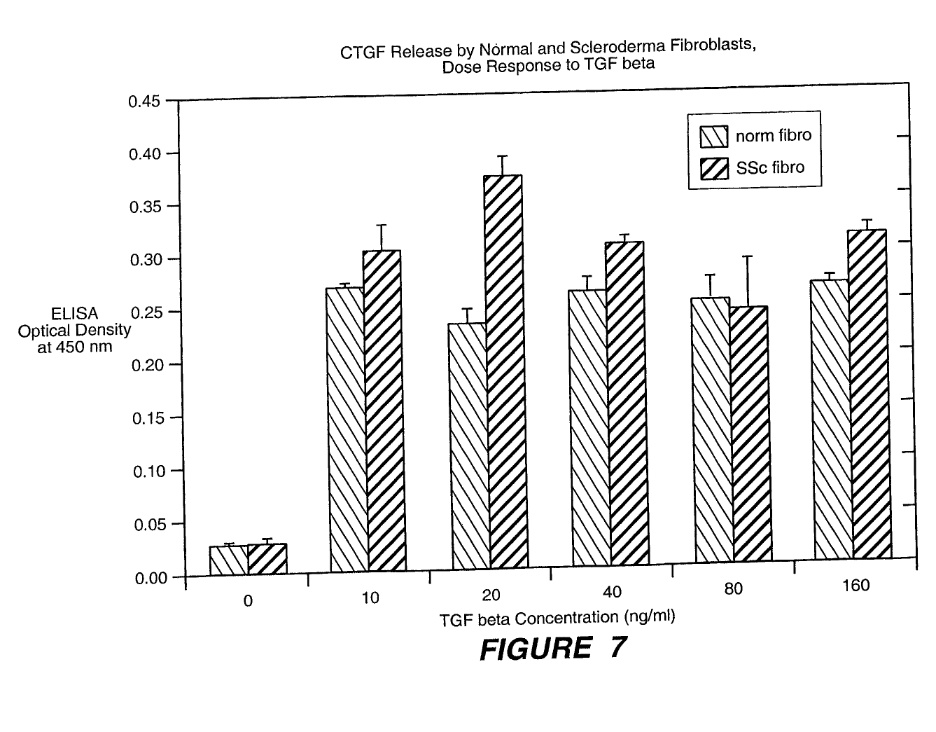

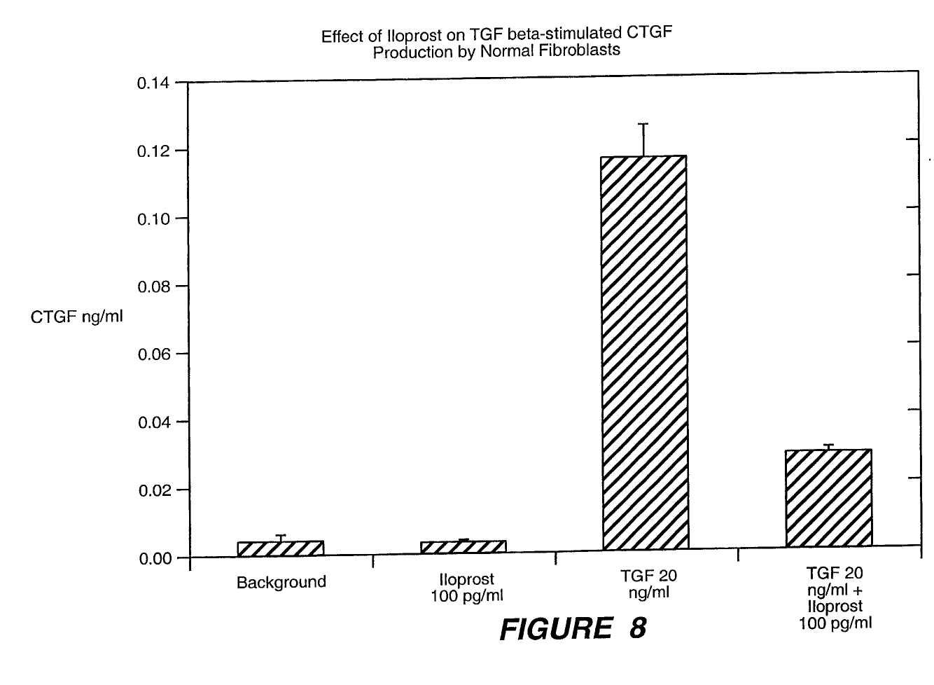

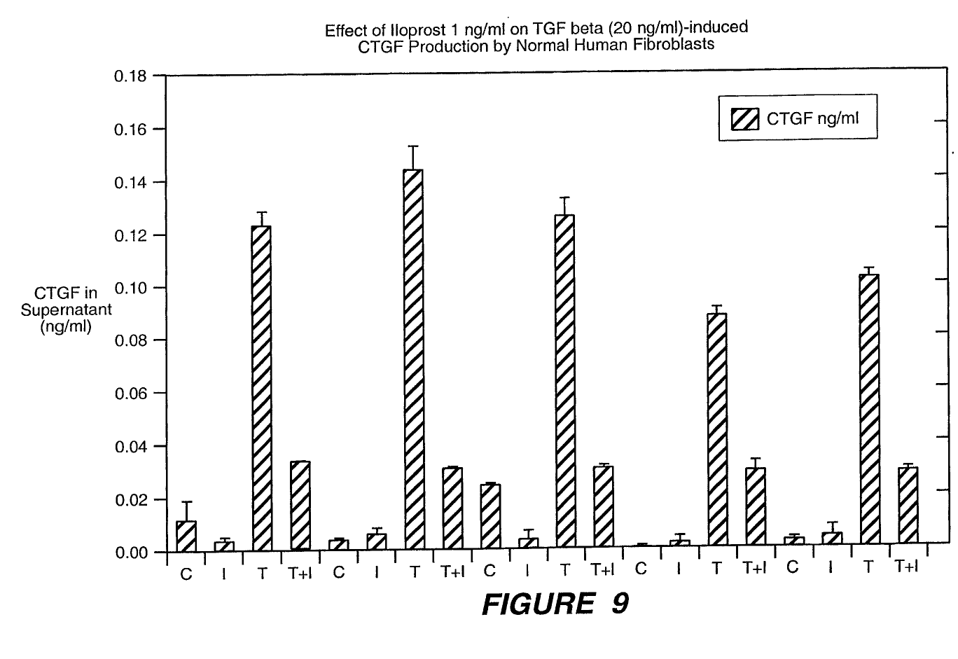

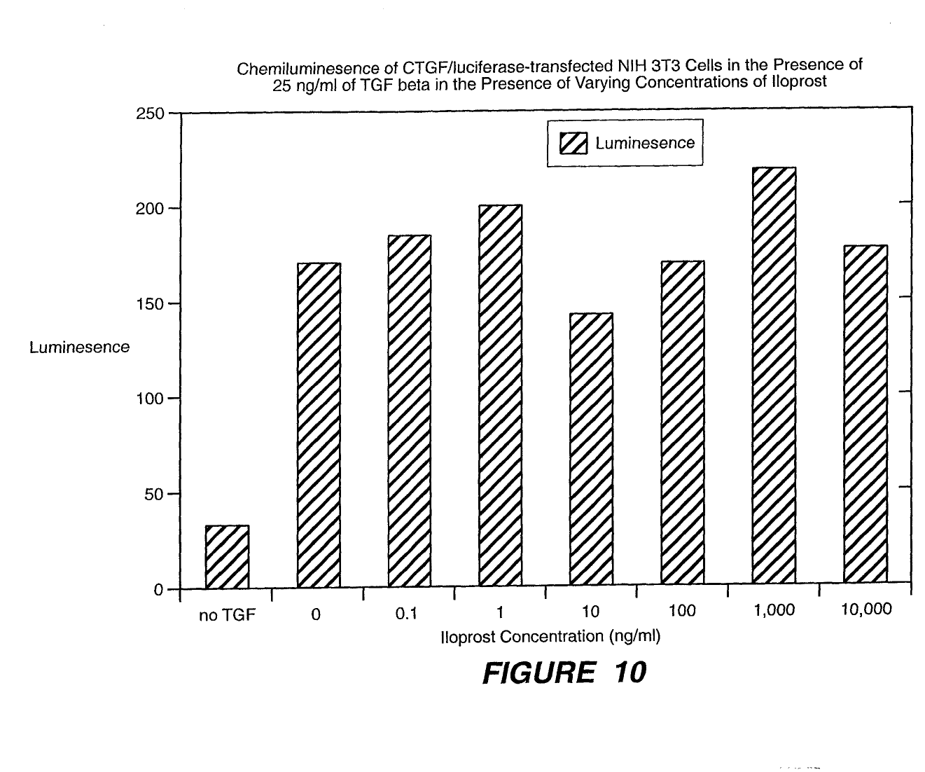

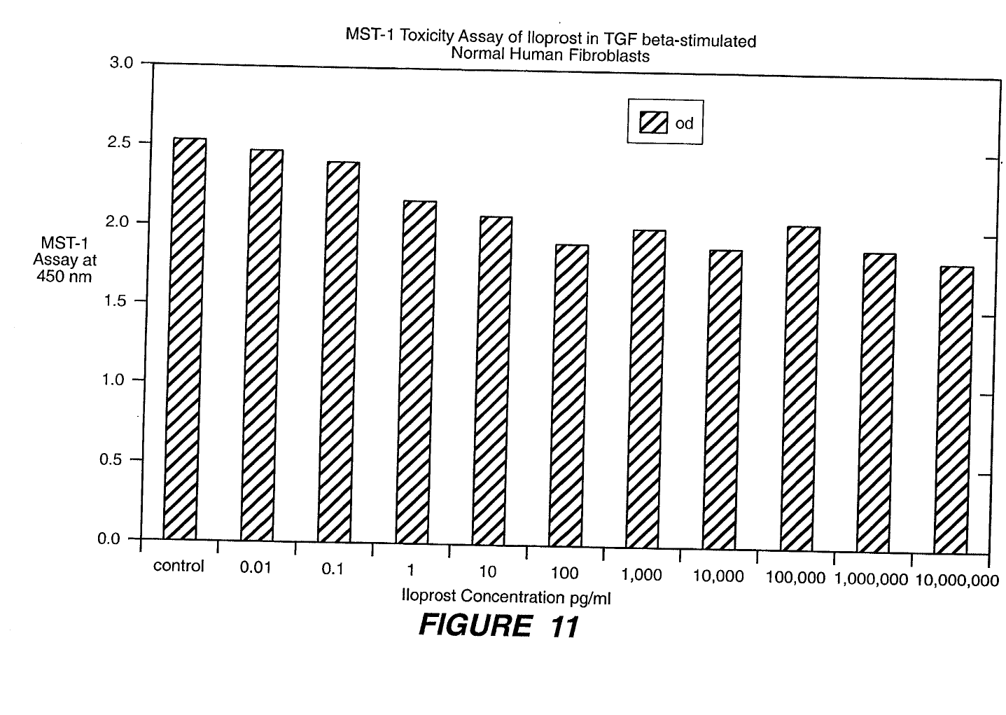

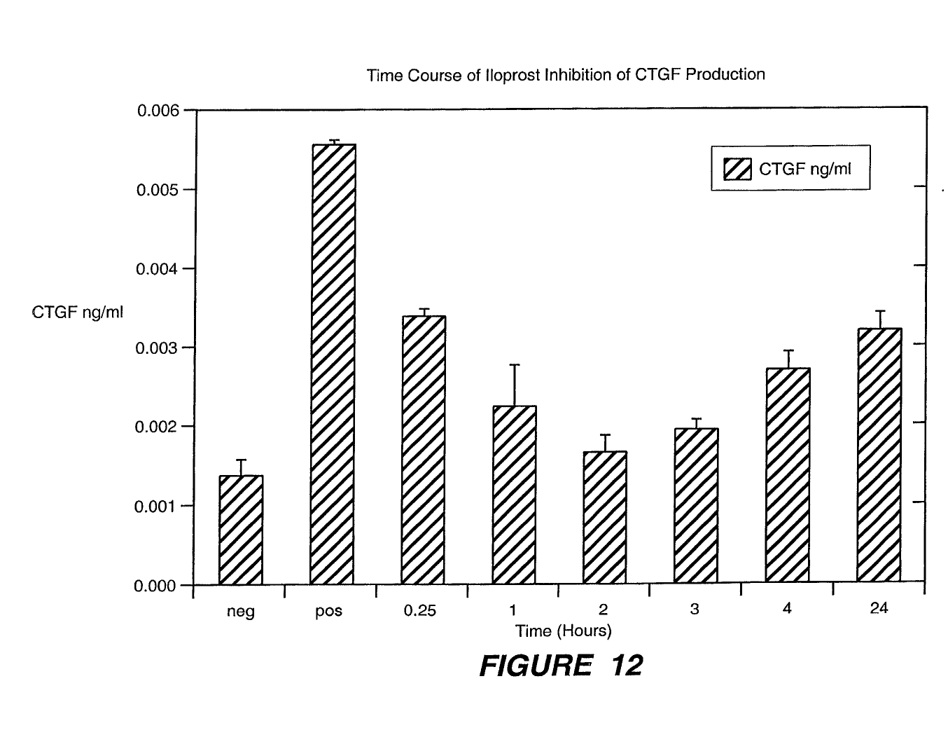

5. Northern blot analysis results resulting from the protocol set forth above (Section C.1.h.) confirm the ability of Iloprost to inhibit CTGF production. See, FIGS. 1 and 2.

The present invention is not to be limited in scope by the exemplified embodiments which are intended as illustrations of single aspects of the invention, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

All references cited within the body of the instant specification are hereby incorporated by reference in their entirety. In addition, the publications listed below are of interest in connection with various aspects of the invention and are incorporated herein as part of the disclosure:

What is claimed:

1. A method for treating a fibrotic disorder comprising administering to a patient in need a therapeutically effective amount of a prostacyclin derivative.

2. The method of claim 1 wherein said fibrotic disorder is scleroderma.

3. The method of claim 1 wherein said prostacyclin derivative is cicaprost.

4. The method of claim 1 wherein said prostacyclin derivative is iloprost.

5. A method for ameliorating the fibrosis related symptoms of scleroderma comprising administering to a patient in need a therapeutically effective amount of a prostacyclin derivative.

6. The method of claim 7 wherein said prostacyclin derivative is cicaprost.

7. The method of claim 7 wherein said prostacyclin derivative is iloprost.
